(19) United States
(12) Patent Application Publication
(10) Pub. No.: US 2019/0167694 A1
(43) Pub. Date: Jun. 6, 2019

(54) NEW THERAPEUTICS USES

(71) Applicant: NOVARTIS AG, BASEL (CH)

(72) Inventors: Yong Jia, San Diego, CA (US); GERALD LELAIS, San Diego, CA (US); THOMAS HERBERT MARSILJE, III, San Diego, CA (US); ANDREW TODD MILLER, San Diego, CA (US); SHAILAJA KASIBHATLA, San Diego, CA (US)

(21) Appl. No.: 16/142,133

(22) PCT Filed: Apr. 5, 2017
(86) PCT No.: PCT/IB2017/051938

§ 371 Date: Sep. 26, 2018

Related U.S. Application Data
(60) Provisional application No. 62/319,981, filed on Apr. 8, 2016.

(51) Int. Cl. A61K 31/55

(52) U.S. Cl. CPC A61K 31/55

(57) ABSTRACT

New uses of an EGFR inhibitor are disclosed. Methods of treatment using the EGFR inhibitor are also disclosed.

### FIELD OF THE INVENTION

The present invention relates to uses of an EGFR inhibitor and to methods of treatment of using the EGFR inhibitor. The present invention also relates to pharmaceutical compositions comprising an EGFR inhibitor for use in the treatment of T cell-mediated autoimmune diseases. This invention also relates to an EGFR inhibitor for use in in the treatment of T cell-mediated autoimmune diseases, to the use of the EGFR inhibitor for the preparation of a medicament for the treatment or prevention of T cell-mediated autoimmune diseases, methods of treating or preventing a T cell-mediated autoimmune disease in a subject in need thereof comprising administering to said subject a therapeutically effective amount of the EGFR inhibitor, and the use of such an EGFR inhibitor for the treatment or prevention of a T cell-mediated autoimmune disease.

### BACKGROUND

The TEC-family of protein tyrosine kinases ITK (inducible T-cell kinase, also known as Itk), RLK and TEC are key components of T-cell-receptor signaling that contribute to the regulation and polarization of T-cell activation. Functional studies have implicated TEC kinases as important mediators of pathways that control CD4⁻ T helper cell differentiation and promote effector functions.

ITK has been shown to regulate autoreactive T cell trafficking. ITK-deficient T cells exhibit reduced proliferative capacity, cytokine secretion, as well as defects in T cell differentiation (reviewed in Andreotti, A. H., et al., T-cell signaling regulated by the Tec family kinase, Itk. Cold Spring Harb Perspect Biol, 2010. 2(7): p. a002287). It was recently demonstrated that the absence of ITK is protective in a mouse model of multiple sclerosis, EAE, with fewer pathogenic T cells developing (Kannan, A. K., et al., Itk signals promote neuroinflammation by regulating CD4+ T-cell activation and trafficking. J Neurosci, 2015. 35(1): p. 221-33. Small molecule inhibitors of ITK were also found to reduce T cell infiltration and destruction of islet cells in Type I Diabetic models-highlighting the potential for ITK inhibitors to treat T-cell mediated autoimmune diseases such as Type I diabetes (Jain, N., et al., CD28 and ITK signals regulate autoreactive T cell trafficking. Nat Med, 2013. 19(12): p. 1632-7). Furthermore, a recent report demonstrated that a small molecule inhibitor of ITK and RLK prevented colitis progression in an adoptive transfer mouse model (Cho, H. S., et al., A Small Molecule Inhibitor of ITK and RLK Impairs Th1 Differentiation and Prevents Colitis Disease Progression. J Immunol, 2015, 195(10): p. 4822-31).

Thus, TEC kinases, and ITK in particular, have now emerged as important modulators of T-cell function that have exciting therapeutic potential for the regulation of autoreactive T-cell responses (see also WO2015054612A1, and references cited therein).

About 10-15% of NSCLC (Non-small cell lung cancer) have activating EGFR mutations and respond to first and second generation EGFR inhibitors, but these inhibit wt (wild-type) EGFR leading to GI (gastrointestinal) toxicity and rash, which compromises clinical utility. In addition, durability of response is limited by acquired resistance, with a secondary mutation at the gatekeeper residue (T790M) occurring in roughly half the cases. COMPOUND A, also known as and referred to herein as EGF816, is a potent third generation, irreversible inhibitor of mutant EGFR (including T790M double mutants) with good selectivity vs. wt EGFR. COMPOUND A is also known as nazartinib. Preclinically, COMPOUND A demonstrated in vivo efficacy in multiple mutant EGFR settings. COMPOUND A is currently undergoing clinical trials, e.g. in advanced non-small cell lung cancer (NSCLC) harboring T790M.

Unexpectedly, COMPOUND A was found to exhibit cross-reactivity with the TEC family of kinases, and in particular, ITK. Consistent with the biology of ITK, COMPOUND A resulted in a block in T cell proliferation in in vitro assays. COMPOUND A may therefore be useful in treating various T-cell mediated autoimmune diseases. Examples of T-cell mediated autoimmune diseases include, in particular, ulcerative colitis, rheumatoid arthritis, Myasthenia gravis, Hashimoto's thyroiditis, polymyositis, Type I diabetes, celiac disease and multiple sclerosis. COMPOUND A may therefore also be useful in treating T-cell mediated autoimmune diseases such as ulcerative colitis, Myasthenia gravis, Hashimoto's thyroiditis, polymyositis, celiac disease, N-infectious uveitis, Sjögren's syndrome, primary biliary cirrhosis, autoimmune hepatitis and ankylosing spondylitis.

### SUMMARY

The present disclosure therefore provides
COMPOUND A, (also known as nazartinib), which is the compound of formula (I) depicted below,

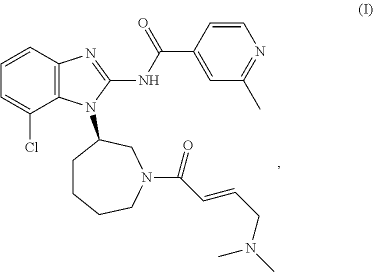 [SMILES: Cc1cc(C(=O)Nc2nc3cccc(Cl)c3n2[C@@H]2CCCCN(C(=O)/C=C/CN(C)C)C2)ccn1]

or a pharmaceutically acceptable salt thereof, for use in treating certain T cell-mediated autoimmune diseases.
The chemical name of COMPOUND A is (R,E)-N-(7-chloro-1-(1-(4-(dimethylamino)but-2-enoyl)azepan-3-yl)-1H-benzo[d]imidazol-2-yl)-2-methylisonicotinamide.

Such a T cell mediated autoimmune disease may be selected from ulcerative colitis, rheumatoid arthritis, Myasthenia gravis, Hashimoto's thyroiditis, polymyositis, Type I diabetes, celiac disease multiple sclerosis, N-infectious uveitis, Sjögren's syndrome, primary biliary cirrhosis, autoimmune hepatitis and ankylosing spondylitis.

The T cell mediated autoimmune disease may also be selected from rheumatoid arthritis, Myasthenia gravis, Hashimoto's thyroiditis, polymyositis, Type I diabetes, celiac disease multiple sclerosis, N-infectious uveitis, Sjögren's syndrome, primary biliary cirrhosis, autoimmune hepatitis and ankylosing spondylitis.

The T cell mediated autoimmune disease may also be selected from ulcerative colitis, Myasthenia gravis, Hashimoto's thyroiditis, polymyositis, celiac disease, N-infectious uveitis, Sjögren's syndrome, primary biliary cirrhosis, autoimmune hepatitis and ankylosing spondylitis.

T cell mediated autoimmune diseases which may be treated by nazartinib include ulcerative colitis, Myasthenia gravis, Hashimoto's thyroiditis, polymyositis, celiac disease, N-infectious uveitis, Sjögren's syndrome, primary biliary cirrhosis, and ankylosing spondylitis.
Other embodiments of the T cell mediated autoimmune disease are as defined in the claims.

Preferably the disease to be treated is Type I diabetes.

The present disclosure therefore also provides:
a pharmaceutical composition comprising COMPOUND A, or a pharmaceutically acceptable salt thereof, for use in treating a T cell-mediated autoimmune disease as defined herein;
a method for the treatment of a T cell-mediated autoimmune disease as defined herein, comprising administering a therapeutically effective amount of COMPOUND A, or a pharmaceutically acceptable salt thereof, to a subject in need thereof;

and
the use of COMPOUND A, or a pharmaceutically acceptable salt thereof, for the manufacture of a medicament for the treatment of a T cell-mediated autoimmune disease as defined herein.

The invention also provides COMPOUND A for use in the treatment of Type I diabetes.

### DETAILED DESCRIPTION

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

As used herein, the terms “treat”, “treatment” and “treating” refer to the reduction or amelioration of the progression, severity and/or duration of a disorder, e.g., an autoimmune disorder, or the amelioration of one or more symptoms (preferably, one or more discernible symptoms) of the disorder resulting from the administration of one or more therapies. In specific embodiments, the terms “treat,” “treatment” and “treating” refer to the amelioration of at least one measurable physical parameter of an autoimmune disorder, not necessarily discernible by the patient. In other embodiments the terms “treat”, “treatment” and “treating”—refer to the inhibition of the progression of an autoimmune disorder, either physically by, e.g., stabilization of a discernible symptom, physiologically by, e.g., stabilization of a physical parameter, or both.

### COMPOUND A

COMPOUND A, also known as nazartinib, is a targeted covalent irreversible inhibitor of Epidermal Growth Factor Receptor (EGFR) that selectively inhibits activating and acquired resistance mutants (L858R, ex19del and T790M), while sparing WT EGFR. (see Jia et al, Cancer Res Oct. 1, 2014 74; 1734). COMPOUND A has shown significant efficacy in EGFR mutant (L858R, ex19del and T790M) cancer models (in vitro and in vivo) with no indication of WT EGFR inhibition at clinically relevant efficacious concentrations.
COMPOUND A is (R,E)-N-(7-chloro-1-(1-(4-(dimethylamino)but-2-enoyl)azepan-3-yl)-1H-benzo[d]imidazol-2-yl)-2-methylisonicotinamide which is disclosed in PCT Publication No. WO 2013/184757. This publication also discloses its method of preparation and pharmaceutical compositions comprising COMPOUND A. A particularly useful salt of COMPOUND A is the mesylate salt or the hydrocholoride thereof. These salts and pharmaceutical compositions thereof are disclosed in WO/2015/083059.

The Examples below are set forth to aid in the understanding of the disclosures but are not intended to, and should not be construed to limit its scope in any way.

### EXAMPLE 1

### COMPOUND A Shows Inhibition on TEC Kinases

T-cell TEC family kinases (ITK, TEC and TXK) are downstream of TCR (T-cell receptor) and play an important role in T-cell activation and signaling. ITK is the most dominant player among TEC family kinases. T-cell hyper-activation has been implicated in many auto-immune diseases, thus inhibition of TEC family kinases might be effective in treating T-cell mediated auto-immune diseases.

COMPOUND A, a potent inhibitor of mutant EGFR, also displays potent inhibition of Tec family kinases in vitro. As shown in Table 1, in the biochemical based assay, COMPOUND A showed single digit nM potency on the three T-cell Tec family members: ITK, TEC and TXK. In the cellular assays, COMPOUND A potently inhibited T-cell Tec family members with IC₅₀ values of 21, 107 and 140 nM in IL2-production, mouse CD4 T-cell and human CD4 T-cell proliferation, respectively. In contrast, COMPOUND A was less potent on B-cell Tec family kinases, as demonstrated by up-shifted IC50 values in mouse B-cell and TMD-8 (BTK-dependent) proliferation assays.

As shown below, COMPOUND A preferentially inhibits T-cells with selectivity over B-cells, whereas ibrutinib is more potent on B-cells than T-cells. Thus ibrutinib has the potential to be more broadly immunosuppressive, whereas COMPOUND A may be selective for T-mediated autoimmune diseases.

In Vitro Assay Methods (Assays Described in Table 1):

The biochemical assays for ITK, TEC and TXK were carried out using Caliper Life Sciences' proprietary LabChip™ technology. This technology uses a microfluidic chip to measure the conversion of a fluorescent peptide substrate to a phosphorylated product. The product conversions were determined in the presence of various compound concentrations and IC₅₀ values were calculated.

The cellular IL-2 Production assay was carried out using Jurkat cells. Upon CD3/CD28 stimulation overnight in the presence of various concentrations of compound, the IL-2 content in the conditioned media was measured by ELISA, and compound (COMPOUND A or ibrutinib) IC₅₀ was determined.

In the Mouse CD4 T cell assay, CD4+ T cells were purified from mouse spleens, and plated in the tissue culture plates coated with anti-CD3. Cells were incubated for 48 h at 37° C. in the presence of various concentrations of compound. ³H-Thymidine was then added and cells were incubated for an additional 18 h at 37° C. Cells were then harvested and read on a beta counter.

In the Human CD4 T cell assay, primary human CD4+ T cells isolated from a leukopak were cultured in the presence of anti-CD³/anti-CD28 beads to stimulate T cell proliferation. After 4 days, cell viability was measured using Cell Titer Glo.

In the Mouse B cell assay, B cells are purified from mouse splenocytes and plated in the tissue culture plates with supplement of anti-IgM and m-IL4. Cells were incubated at 37° C. in the presence of various concentrations of compound. After 3 days, cell viability was measured using Cell Titer Glo.

In the BTK-dependent TMD-8 cell proliferation assay, TMD-8 cells were incubated at 37° C. in the presence of various concentrations of compound. After 3 days, cell viability was measured using Cell Titer Glo.

TABLE 1
Biochemical and cellular IC₅₀ on Tec family
kinases of COMPOUND A and ibrutinib

| | | COMPOUND A | Ibrutinib |
|---|---|---|---|
| Type | Assay | IC₅₀ (nM) | IC₅₀ (nM) |
| Cellular | IL2-Production* | 21 | 284 |
| | Mouse CD4 T cell | 107 | 1600 |
| | Human CD4 T cell | 140 | ND |
| | Mouse B cell | 295 | 36 |
| | BTK (TMD-8) | 224 | 2 |
| Biochemical | ITK | 1.3 | 11 |
| | TEC | 0.9 | 78 |
| | TXK | 2 | ND |

*IL2-production assay encompasses the TEC-family kinases (ITK, TEC, and TXK)

### EXAMPLE 2

### Immune-Modulatory In Vivo Effect of COMPOUND A

T-cells play critical roles in immune regulation. T-cell Tec family kinases are important players in T-cell function, which in turn can modulate immune function. COMPOUND A was tested in a T-cell dependent antibody response (TDAR) assay, a frequently used functional assessment of the immune system. COMPOUND A was administered orally to rats for 5 weeks at a dose of 30 mg/kg/day. On study days 11 and 25 for the main study animals and days 28 and 42 for the recovery group, animals received 300 μg of KLH (Keyhole Limpet Hemocyanin) antigen. Samples for serology assessment of anti-KLH IgM and anti-KLH IgG antibodies (study days 19, 21, 23, 25 and 36 prior to dosing from the main study animals; recovery days 42 and 53 prior to KLH injection from the recovery animals) were collected. Immunomodulatory responses in COMPOUND A-treated animals following KLH immunization were noted when values were compared to concurrent vehicle controls. As shown in Table 2, the decrease in anti-KLH IgM antibodies (primary response) peaked on study day 19-21 for all test groups in both male and female rats. The decreases were also observed in mean anti-KLH IgM values on study days 21, 23, 25 (primary response, time course) and day 36 (post boosting) for female rats. For anti-KLH IgG antibodies, decreases in mean concentration were apparent on study days 19, 21, 23 and 25 for both male and female rats. On study day 36, decreases in mean concentration were detected in female rats. Recovery following withdrawal of COMPOUND A treatment was noted. The COMPOUND A-related decrease of anti-KLH antibody production in both male and female rats was reversible. This included both primary response-anti-KLH IgM, and isotype switch measured by secondary anti-KLH IgG production as indicated by values that were similar to concurrent controls at the recovery sampling time points (recovery day 42 and 53).

In summary, the in vivo effect of COMPOUND A on primary IgM antibody formation and the isotype switch to IgG antibody was noted at 30 mg/kg. This effect was reversed following withdrawal of COMPOUND A. Together, the in vitro biochemical/cellular data and in vivo TDAR results indicate that COMPOUND A has potential immune-modulatory potential.

TABLE 2
Decreased anti-KLH IgM and IgG indicated by mean percent
differences compared to vehicle controls following
administration of COMPOUND A at 30 mg/kg/day

| | |
|---|---|
| Males | Female |

| Study Days | IgM | IgG | IgM | IgG |
|---|---|---|---|---|
| 19 | −67 | −78 | −88 | −96 |
| 21 | — | −84 | −75 | −98 |
| 23 | — | −80 | −71 | −97 |
| 25 | — | −83 | −63 | −96 |
| 36 | — | — | −67 | −69 |
| 42* | — | — | — | — |
| 53* | — | — | — | — |

‘—’ indicates results were not considered meaningfully different from control results.
Mean % difference = (mean dose group value − mean control value)/mean control value) × 100%
*indicates recovery days

Together, the in vitro biochemical/cellular data and in vivo TDAR results indicate that COMPOUND A inhibits T cell functions.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject disclosure have been discussed, the above specification is illustrative and not restrictive. The full scope of the disclosure should be determined by reference to the claims, along with their full scope of equivalents, and the specification, along with such variations.

1-2. (canceled)

3. A method for the treatment of a T cell-mediated autoimmune disease comprising administering a therapeutically effective amount of the compound (R,E)-N-(7-chloro-1-(1-(4-(dimethylamino)but-2-enoyl)azepan-3-yl)-1 H-benzo[d]imidazol-2-yl)-2-methylisonicotinamide (nazartinib), or a pharmaceutically acceptable salt thereof, to a subject in need thereof.

4. (canceled)

5. The method according to claim 3, wherein the T cell mediated autoimmune disease is selected from ulcerative colitis, rheumatoid arthritis, Myasthenia gravis, Hashimoto's thyroiditis, polymyositis, Type I diabetes, celiac disease, multiple sclerosis, N-infectious uveitis, Sjögren's syndrome, primary biliary cirrhosis, autoimmune hepatitis and ankylosing spondylitis.

6. The method according to claim 3, wherein the T cell mediated autoimmune disease is selected from rheumatoid arthritis, Myasthenia gravis, Hashimoto's thyroiditis, polymyositis, Type I diabetes, celiac disease, multiple sclerosis, N-infectious uveitis, Sjögren's syndrome, primary biliary cirrhosis, autoimmune hepatitis and ankylosing spondylitis.

7. The method according to claim 3, wherein the T cell mediated autoimmune disease is selected from ulcerative colitis, Myasthenia gravis, Hashimoto's thyroiditis, polymyositis, celiac disease, N-infectious uveitis, Sjögren's syndrome, primary biliary cirrhosis, autoimmune hepatitis and ankylosing spondylitis.

8. The method according to claim 3, wherein the T cell mediated autoimmune disease is selected from ulcerative colitis, Myasthenia gravis, Hashimoto's thyroiditis, polymyositis, celiac disease, N-infectious uveitis, Sjögren's syndrome, primary biliary cirrhosis, and ankylosing spondylitis.

9. The method according to claim 3, wherein the T cell mediated autoimmune disease is ulcerative colitis.

10. The method according to claim 3, wherein the T cell mediated autoimmune disease is Myasthenia gravis.

11. The method according to claim 3, wherein the T cell mediated autoimmune disease is Hashimoto's thyroiditis.

12. The method according to claim 3, wherein the T cell mediated autoimmune disease is polymyositis.

13. The method according to claim 3, wherein the T cell mediated autoimmune disease is celiac disease.

14. The method according to claim 3, wherein the T cell mediated autoimmune disease is N-infectious uveitis.

15. The method according to claim 3, wherein the T cell mediated autoimmune disease is Sjögren's syndrome

16. The method according to claim 3, wherein the T cell mediated autoimmune disease is primary biliary cirrhosis.

17. The method according to claim 3, wherein the T cell mediated autoimmune disease is ankylosing spondylitis.

18. The method according to claim 3, wherein the T cell mediated autoimmune disease is autoimmune hepatitis.

19. The method according to claim 3, wherein the T cell mediated autoimmune disease is Type I diabetes.

20. The method according to claim 3, wherein the T cell mediated autoimmune disease is multiple sclerosis.

21. The method according to claim 3, wherein the T cell mediated autoimmune disease is rheumatoid arthritis.
